(19) United States
(12) Patent Application Publication
(10) Pub. No.: US 2022/0152584 A1
(43) Pub. Date: May 19, 2022

(54) GAS-TIGHT, HEAT-PERMEABLE MULTILAYER CERAMIC COMPOSITE TUBE

(71) Applicant: BASF SE, Ludwigshafen am Rhein (DE)

(72) Inventors: Grigorios Kolios, Ludwigshafen (DE); Heinrich Laib, Ludwigshafen (DE); Frederik Scheiff, Ludwigshafen (DE); Bernd Zoels, Ludwigshafen (DE); Matthias Kern, Ludwigshafen (DE)

(21) Appl. No.: 17/439,540

(22) PCT Filed: Mar. 6, 2020
(86) PCT No.: PCT/EP2020/056003

§ 371 Date: Sep. 15, 2021

(30) Foreign Application Priority Data
Mar. 15, 2019 (EP) 19163157.1

(51) Int. Cl. B01J 21/04; B32B 1/08; B32B 18/00; B32B 3/30; B01D 69/02; B01D 69/04; B01D 69/12; B01D 71/02; B01J 27/224; B01J 21/18; B01J 21/12; B01J 35/06; B01J 35/00; C01B 3/16; C01B 3/26; C01B 32/05; C01C 3/02; C10G 9/36; C07C 2/82; C10G 9/20; F16L 9/14; F16L 9/10

(52) U.S. Cl. CPC B01J 21/04; C01B 2203/1082; B32B 18/00; B32B 3/30; B01D 69/02; B01D 69/04; B01D 69/12; B01D 71/025; B01D 71/021; B01J 27/224; B01J 21/18; B01J 21/12; B01J 35/06; B01J 35/002; C01B 3/16; C01B 3/26; C01B 32/05; C01C 3/0208; C10G 9/36; C07C 2/82; C10G 9/203; F16L 9/14; F16L 9/10; B32B 2597/00; B32B 2307/732; B32B 2262/10; B01D 2325/02; B01D 2325/04; B01D 2325/06; B01D 2325/22; C01B 2203/0283; B32B 1/08

(57) ABSTRACT

Described herein is a gaslight multilayered composite tube having a heat transfer coefficient of >500 W/m²/K which in its construction over the cross section of the wall of the composite tube includes as an inner layer a nonporous monolithic oxide ceramic surrounded by an outer layer of oxidic fiber composite ceramic, where this outer layer has an open porosity of 5%<ε<50%, and which on the inner surface of the composite tube includes a plurality of depressions oriented towards the outer wall of the composite tube. Also described herein is a method of using the multilayered composite tube as a reaction tube for endothermic reactions, jet tubes, flame tubes or rotary tubes.

The present invention relates to a gastight multilayered composite tube or regions of a multilayered composite tube having a heat transfer coefficient of >500 W/m²/K comprising at least two layers which in its construction over the cross section of the wall of the composite tube comprises as an inner layer a nonporous monolithic oxide ceramic surrounded by an outer layer of oxidic fiber composite ceramic, wherein this outer layer has an open porosity c (according to DIN EN 623-2) of more than 5% and less than 50%, preferably more than 10% and less than 30%, and the inner surface of the composite tube comprises a plurality of depressions oriented towards the outer wall of the composite tube.

Endothermic reactions are often at the start of the value chain in the chemical industry, for example in the thermal cracking of ethane, propane, butane, naphtha and high-boiling crude oil fractions, the reforming of natural gas, the dehydrogenation of propane, the dehydroaromatization of methane to afford benzene or the pyrolysis of hydrocarbons. These reactions are highly endothermic and proceed at high temperatures, i.e. temperatures between 500° C. and 1700° C. are necessary to achieve industrially and economically significant yields.

For example thermal cracking of hydrocarbons, so-called steamcracking, comprises parallel endothermic cracking and dehydrogenation reactions performed at a slight positive pressure and high temperatures. The standard process according to the prior art is steam reforming of ethane, propane or naphtha (unhydrogenated straight-run gasoline) for producing ethylene, propylene and C4 olefins. These products are counted among the quantitatively most important precursors in the chemical industry. Steamcrackers are among the chemical plants with the greatest mass throughput. In the prior art this highly endothermic process is performed in tube coils which are externally heated by firing. In a so-called cracking furnace several parallel tubes are simultaneously heated and internally traversed by a feedstock/steam mixture. The function of the tube walls is the transfer of the heat flow from an external heat source into the reaction volume and the hermetic separation of the reaction volume from the surrounding heat source to maintain the pressure difference between the two spaces. The tubes of the fixed bed reactors are typically cylindrical with a variable or uniform diameter over the entire tube length. Various tubes may also be divided or combined inside the furnace. The material of the tubes is typically a highly alloyed austenitic centrifugal casting.

Industrial cracking processes are performed at pressures up to 5 bar positive pressure and temperatures up to 1000° C., wherein this value represents the product gas temperature at the exit of the reaction tubes. The industrial process is especially kinetically limited. The term “kinetically limited” is to be understood as meaning that the residence time of the reaction gas in the cracking tubes is so short that the cracking and the dehydrogenation reactions do not achieve thermodynamic equilibrium.

When using metallic reactor materials the maximum outer tube wall temperature is limited to about 1050-1100° C.

However, a higher maximum outer tube wall temperature is desirable for many reasons but especially to provide the necessary heat for the cracking reactions despite coke deposits on the tube interior. Coke deposits on the hot tube wall firstly have the result that the outer tube wall temperature must be increased in the course of operation to compensate the thermal insulation effects of the coke. This results in a higher firing output and higher energy consumption. Secondly, coke deposits have the result that upon achieving the maximum allowable outer tube wall temperature the furnace must be taken out of service and decoked by flame cleaning with air.

Tube wall temperatures of >1100° C. necessitates the use of ceramic materials, preferably of oxide ceramics. The advantages of ceramic materials, in particular oxide ceramics are a high heat resistance up to 1800° C., chemical passivity, corrosion resistance and high strength. The greatest disadvantage of ceramic materials is their great brittleness. This property is described by the fracture toughness K_{IC} which is determined for example according to DIN EN ISO 12737 for metals and according to DIN EN ISO 15732 for monolithic ceramics. For steel, a representative of tough materials K_{IC} is ≈50 MPa √m. For monolithic ceramics, for example zirconium oxide (ZrO₂) or corundum (Al₂O₃) K_{IC} is ≅3-5 MPa √m. This makes monolithic ceramics unsuitable for pressure apparatuses having a pressure of >0.5 bar since these materials cannot ensure the “crack before fracture” criterion but may instead be affected by a sudden unannounced fracture.

One alternative is provided by fiber composite ceramics composed of oxidic fibers embedded in a porous matrix of oxidic ceramic. The open porosity c of fiber composite ceramics may generally assume values between 5% and 50%. The advantages of fiber composite ceramics are high heat resistance to 1300° C. or more, high thermal shock resistance and a pseudo-ductile deformation and fracture behavior. The fracture toughness of fiber composite ceramics can attain values of K_{IC}≅10 50 MPa √m. As a result of their porous structure fiber composite ceramics have a relatively low density, a relatively low modulus of elasticity and a relatively low thermal conductivity compared to monolithic ceramics having the same chemical composition. Table 1 comprises a list of the relevant standards for determining these parameters.

TABLE 1
List of relevant standards for determining structural, mechanical
and thermophysical parameters for monolithic ceramics
and composite ceramics

| Parameter | Monolithic ceramic | Fiber composite ceramic |
|---|---|---|
| Density, open porosity | DIN EN 623-2 | DIN V ENV 1389 |
| Elastic modulus | DIN V ENV 843-2 | DIN EN 658-1 |
| Fracture toughness¹ | DIN EN ISO 15732 | Single-edge notch bend² |
| Thermal diffusivity | DIN EN 821-2 | DIN V ENV 1159-2 |
| Specific heat capacity | DIN EN 821-3 | DIN V ENV 1159-3 |

¹The fracture toughness of metallic materials is determined according to DIN EN ISO 12737.
²M. Kuntz. Crack resistance of ceramic fiber composite materials. Dissertation, Karlsruhe University, Shaker Verlag, 1996.

Thermal conductivity is defined by the following relationship:

Thermal conductivity=density×(specific heat capacity)×thermal diffusivity

By way of example, table 2 comprises a comparison between the properties of monolithic ceramics and fiber composite ceramics based on aluminum oxide.

TABLE 2
Comparison of physical properties of monolithic
ceramics and composite ceramics

| | | Fiber composite |
|---|---|---|
| | Monolithic ceramic | ceramic |
| | Friatec Degussit ® | WHIPOX ® |
| Parameter | AL23 | N610/45 |
| Open porosity in % | 0 | 26 |
| $Density in \frac{g}{{cm}^{3}}$ | 3.8 | 2.9 |
| Elastic modulus in GPa | 380 | 110 |
| $Thermal conductivity in \frac{W}{m \cdot K}$ | 30 (@ 100° C.) 5.5 (@ 1000° C.) | 5.7 (@ 200° C.) 2.7 (@ 1000° C.) |

A disadvantage of the porous structure of fiber composite ceramics is their unsuitability for the production of high-pressure apparatuses having a pressure of >0.5 bar. The poorer thermal conductivity compared to nonporous monolithic ceramic having the same chemical composition is a further disadvantage, i.e. when a heat flow is to be transferred through a layer of this material.

WO 2016/184776 A1 discloses a multilayered composite tube comprising a layer of nonporous monolithic oxide ceramic and a layer of oxidic fiber composite ceramic which is employable for producing reaction tubes operated at operating pressures of 1 to 50 bar and the reaction temperatures up to 1400° C. and are thus intensively heated by an external heat source—typically a heating chamber.

However, in operation of these composite tubes undesired solid deposits may be formed on the composite tube inner wall, thus impairing heat transfer and therefore the efficiency of the process to such an extent that the oven must be periodically decoked by flame cleaning. Even complete blockage of the free tube cross section in the interior of the composite tube may occur in extreme cases. Such solid deposits may be formed for example by side reactions of hydrocarbons to form solid carbon in the production of synthesis gas by reforming of hydrocarbons with steam and/or carbon dioxide, in the coproduction of hydrogen and pyrolysis carbon by pyrolysis of hydrocarbons, in the production of hydrocyanic acid from methane and ammonia or from propane and ammonia, in the production of olefins by steamcracking of hydrocarbons and/or coupling of methane to ethylene, acetylene and to benzene. Such carbon deposits are widely referred to as coke. In common with other industrial cokes they are formed by high temperature treatment of an at least partially hydrocarbon-containing substance in a low-oxygen or oxygen-free environment, wherein the low-oxygen refers to an environment in which the oxygen present is insufficient for complete combustion to form CO2 and steam.

In the case of thermal steam cracking of hydrocarbons three different types of cokes are distinguished—firstly so-called catalytic coke which is formed on the catalytically active elements of the tube surface, in particular iron (Fe) and nickel (Ni), secondly pyrolytic coke formed by reactions in the gas phase without interaction with the tube wall, and thirdly condensation coke which is formed by condensation of higher molecular weight hydrocarbons at temperatures in the range 400-600° C. and which is relevant especially to the exit region from the high temperature zone. In the reaction tube itself catalytic and pyrolytic coking dominate.

Attempts at coke prevention have been made as long ago as the 1960s. These include the development of highly-alloyed, austenitic metallic centrifugally cast tubes which are said to form a protective chromium or aluminum oxide layer under process conditions. A second line of development was that of reducing the tube wall temperature by improving the tube interior-side heat transfer to the process fluid. It is an object of these measures to reduce the temperature at the tube wall interior and to retard the catalytic coking reaction occurring there.

Numerous concepts are disclosed in the prior art to improve the transport properties between the gas stream and the tube wall. There are for example tubes having ribs or inserted flow elements running along the axis.

WO 2015/052066 A1 describes a reaction tube for producing hydrogen cyanide which comprises an inserted rib-shaped insert body. This is said to increase the space-time yield. However, this does not effectively counter the risk of undesired deposits at the tube wall.

WO 2017/007649 A1 discloses a reaction tube having depressions. It discloses general explanations thereof and a multiplicity of material embodiments but no indication of multilayered composite tubes having a heat transfer coefficient of >500 W/m²/K which in its construction over the cross section of the wall of the composite tube comprises as an inner layer a nonporous monolithic oxide ceramic surrounded by an outer layer of oxidic fiber composite ceramic and which has an open porosity of 5%<ε<50%.

A person skilled in the art and familiar with WO 2017/007649 A1 would also not have considered the implementation of the depressions according to the invention in such composite tubes since the introduction of depressions into a nonporous monolithic oxide ceramic gave reason to fear that the required strength of the component would no longer be ensured as a result. There was thus reason to fear that the required strength could only be achieved by increasing the wall thickness which would negate the advantage of the introduced depressions due to impairment of the heat transfer.

A person skilled in the art would also have had reason to fear that the introduction of depressions in the case of this material which exhibits a high brittleness would markedly increase the risk of undesired crack formation.

Finally a person skilled in the art would not have introduced depressions into ceramic tubes since these preclude catalytic coke growth and the high fabrication complexity familiar from metallic tubes would thus not have been justifiable by a reduction in pyrolytic coking alone.

WO 2017/178551 A1 likewise describes a reactor for cracking reactions in which the inner wall of the reactor tube comprises depressions (claim 1). This document too discloses general explanations thereof and a multiplicity of material embodiments but no indication of multilayered composite tubes having a heat transfer coefficient of >500 W/m²/K which in its construction over the cross section of the wall of the composite tube comprises as an inner layer a nonporous monolithic oxide ceramic surrounded by an outer layer of oxidic fiber composite ceramic and which has an open porosity of 5%<ε<50%. The implementation of depressions in such composite tubes would not have been considered by a person skilled in the art familiar with WO 2017/178551 A1 either since, similarly to familiarity with WO2017/007649, there would have been reason to fear insufficient strength, excessive brittleness and excessive fabrication complexity.

The problem addressed by the present invention is accordingly that of providing reaction tubes having the following profile of properties: (i) heat-permeable with a heat transfer coefficient

$$> 500 \frac{W}{{m}^{2} K} ,$$

(ii) heat-resistant to >1100° C., (iii) pressure resistant to about 5 bar/stable at pressure differences up to about 5 bar (iv) corrosion-resistant to reducing atmospheres and to oxidizing atmospheres having oxygen partial pressures of 10⁻²⁵ bar to 10 bar (v) thermal shock resistance according to DIN EN 993-11 and (vi) chemically inert toward undesired deposits, in particular inert to coking on the inner wall of the reaction tube catalyzed by metals such as iron and nickel and (vii) heat transfer also improved such that pyrolytic coking is reduced.

Disclosed here is a multilayered composite tube having a heat transfer coefficient of more than 500 W/m²/K comprising at least two layers which in its construction over the cross section of the wall of the composite tube comprises as an inner layer a nonporous monolithic oxide ceramic surrounded by an outer layer of oxidic fiber composite ceramic and which has an open porosity of 5%<ε<50% and which on the inner surface of the composite tube comprises a plurality of depressions oriented towards the outer wall of the composite tube.

The depressions according to the invention may be arranged on the inner wall of the composite tube irregularly or preferably regularly.

The preferred number of the introduced depressions on a specific surface element of the tube inner wall is influenced by the particular technical circumstances. It is generally advantageous for the inner surface of the composite tube to be provided with depressions according to the invention preferably to an extent of 10% to 95%, particularly preferably 50% to 90%. In terms of the depressions reference is made to the proportion by area which the depression respectively occupies directly on the surface of the tube interior.

The shape and the depth of the depressions may be identical or different over the length of the tube inner wall of the composite tube. It may be particularly advantageous for the shape of the depressions according to the invention to be made such that sharp edges in the contour are avoided to instead give a rounded, curved contour such as is the case for example in spheroid, ovoid, spherical, concave or droplet-shaped depressions. More particular indications for a possible shape of such depressions are apparent to a person skilled in the art from WO 2017/178551 A1.

The depressions according to the invention are applied to the tube inner wall of the composite tube oriented towards the outside and are disposed exclusively in the innermost layer of the composite tube which is made of nonporous monolithic oxide ceramic.

A person skilled in the art and familiar with WO 2017/007649 A1 would also not have considered the implementation of the depressions according to the invention in such composite tubes since the introduction of depressions into a nonporous monolithic oxide ceramic gave reason to fear that the required strength of the component would no longer be ensured as a result. There was thus reason to fear that the required strength could only be achieved by increasing the wall thickness which would negate the advantage of the introduced depressions due to impairment of the heat transfer.

A person skilled in the art would also have had reason to fear that the introduction of depressions in the case of this material which exhibits a high brittleness would markedly increase the risk of undesired crack formation.

Finally a person skilled in the art would not have introduced depressions into ceramic tubes since these preclude catalytic coke growth and the high fabrication complexity familiar from metallic tubes would thus not have been justifiable by a reduction in pyrolytic coking alone.

However, it is surprisingly possible to achieve the required properties in the composite tube according to the invention. It is particularly advantageous when the maximum depth of the depressions according to the invention is 0.5-2 mm. As previously mentioned the depth of the depressions may optionally vary within the composite tube. This may be particularly advantageous to allow precise adjustment of the requirements in terms of heat transfer and coking propensity which vary in the flow direction. The preferred configuration in a specific case depends on the particular furnace geometry.

Further indications for a possible shape of such depressions are apparent to a person skilled in the art from WO 2017/178551 A1.

The introduction of the depressions according to the invention may be effected in different ways. They may preferably advantageously be impressed during production of the monolithic ceramic tube by introduction into the soft material after the processing steps of extruding, casting or pressing and before firing. The depressions may advantageously be impressed during production of the monolithic ceramic tube in the step of so-called protoforming by dry, wet or isotactic pressing because the forming and thus the introduction of the depressions is simple in terms of production engineering and may be performed with great geometric degrees of freedom. The impressing of the depressions according to the invention is preferably carried out during protoforming by pressing processes.

The impressing of the depressions according to the invention into the multilayered composite tube may in particular be produced in a manner that is simple and effective in terms of process engineering by press-forming. Compared to metallic materials, produced for example by centrifugal casting or extrusion, this material advantageously provides for the option of forming in the cold state (before firing) and without subtractive methods.

In experiments it has surprisingly been found that the multilayered composite tube according to the invention has a broader temperature distribution of the tube inner wall than a tube of identical geometry and structure based on a metallic material. The relatively low temperature has the result that in the composite tube according to the invention coking is more effectively prevented than in a comparable metallic tube. This would not have been expected by a person skilled in the art.

The two layers in the composite tube according to the invention advantageously adhere to one another through mechanical or atomic-level joins. Relevant mechanical joins are for example pressure fit joins. Relevant atomic-level joins for this invention include adhesive bonding and sintering. All join types belong to the prior art (W. Tochtermann, F. Bodenstein: Konstruktionselemente des Maschinenbaues, part 1. Grundlagen; Verbindungselemente; Gehause, Behalter, Rohrleitungen and Absperrvorrichtungen. Springer-Verlag, 1979).

The wall of the multilayered composite tube advantageously comprises, at least in regions, two layers, namely a layer of nonporous monolithic oxide ceramic and a layer of oxidic fiber composite ceramic; i.e. the multilayered composite tube may also be a composite tube section. This may include for example a composite tube which is zoned or divided into points and composed of two layers only in regions. However, it is preferable when the entire wall of the composite tube which is subjected to an external temperature, for example by a heating chamber, of >1100° C. comprises at least two layers, namely a layer of nonporous monolithic oxide ceramic and a layer of oxidic fiber composite ceramic.

The pipe section of the multilayered composite tube subjected to an external temperature, for example by a heating chamber, of >1100° C. advantageously comprises no metallic layers.

The inner tube is advantageously wrapped with a layer of oxidic fiber composite ceramic. The two layers may be joined to one another by mechanical or atomic-level joins to form a component. The properties of this component are determined by the heat resistance and the deformation behavior of the layer of oxidic fiber composite ceramic. The gastightness is provided by the inner tube of oxide ceramic. When using an oxide-ceramic inner tube the inside of the tube wall has a high chemical stability and abrasion resistance with a hardness >14000 MPa for aluminum oxide, >12000 MPa for zirconium oxide.

At 1400° C. aluminum oxide and magnesium oxide for example are stable over the entire range of oxygen partial pressure from 10⁻²⁵ bar to 10 bar while all other ceramic materials undergo a transition between reduction and oxidation and therefore corrode (Darken. L. S., slurry, R. W. (1953). Physical chemistry of metals. McGraw-Hill).

The tube internal diameter of the multilayered composite tube is advantageously 10 mm to 1000 mm, preferably 10 mm to 100 mm, in particular 40 mm to 80 mm. The total wall thickness of at least two layers is advantageously 0.5 mm to 50 mm, preferably 1 mm to 30 mm, in particular 2 mm to 20 mm. The thickness of the layer of oxidic fiber composite ceramic is advantageously less than 90%, preferably less than 50%, in particular less than 25%, of the total wall thickness; the thickness of the layer of oxidic fiber composite ceramic is advantageously at least 10% of the total wall thickness. The thickness of the layer of monolithic oxide ceramic is advantageously from 0.5 mm to 45 mm, preferably from 1 mm to 25 mm, particularly preferably from 2 mm to 15 mm. The thickness of the layer of oxidic fiber composite ceramic is advantageously from 0.5 mm to 5 mm, preferably from 0.5 mm to 3 mm.

The length of the multilayered composite tube is advantageously 0.5 to 20 m, preferably 1 to 10 m, in particular 1.5 to 7 m. It is possible to join a plurality of such tubes to one another through elbows and/or collectors, wherein these elbows and collectors may optionally also be in the form of multilayered composite moldings and may comprise the depressions according to the invention.

The disclosed multilayered composite tube comprising at least one layer of nonporous monolithic oxide ceramic and at least one layer of oxidic fiber composite ceramic advantageously has an open porosity of 5%<ε<50%, preferably 10%<ε<30%. The multilayered composite tube is particularly advantageously gastight. The term “gastight” is to be understood as meaning a solid having an open porosity according to DIN EN 623-2 of zero. The allowable measurement accuracy is <0.3%.

The density of the nonporous monolithic oxide ceramic is advantageously higher than the density of the oxidic fiber composite ceramic. The density of the nonporous monolithic oxide ceramic is advantageously between

$$1000 \frac{kg}{{m}^{3}} and 7000 \frac{kg}{{m}^{3}} ,$$

in particular between

$$2000 \frac{kg}{{m}^{3}} and 5000 \frac{kg}{{m}^{3}} ,$$

for example

$$2800 \frac{kg}{{m}^{3}}$$

for mullite (about 70% aluminum oxide) or

$$3700 \frac{kg}{{m}^{3}}$$

for aluminum oxide of >99.7% purity. The density of the layer of fiber composite ceramic is between

$$500 \frac{kg}{{m}^{3}} and 3000 \frac{kg}{{m}^{3}} .$$

The ratio of the densities of the monolithic ceramic and the fiber composite ceramic in the composite structure is advantageously between 1:1 and 3:1, in particular between 1:1 and 2:1.

The material-dependent elastic modulus of the nonporous monolithic oxide ceramic is advantageously greater than the elastic modulus of the oxidic fiber composite ceramic. The elastic modulus of the nonporous monolithic oxide ceramic is advantageously between 100 GPa and 500 GPa, in particular between 150 GPa and 400 GPa, for example 150 GPa for mullite (about 70% aluminum oxide) or 380 GPa for aluminum oxide of >99.7% purity. The elastic modulus of the layer of fiber composite ceramic is between 40 GPa and 200 GPa. These values are at 25° C. The ratio of the elastic moduli of the monolithic ceramic and the fiber composite ceramic in the composite structure is advantageously between 1:1 and 5:1, in particular between 1:1 and 3:1.

The material-dependent thermal conductivity of the nonporous monolithic oxide ceramic is advantageously greater than the thermal conductivity of the oxidic fiber composite ceramic. The thermal conductivity of the nonporous monolithic oxide ceramic is advantageously be tween

$$1 \frac{W}{m \cdot K} and 50 \frac{W}{m \cdot K} ,$$

in particular between

$$30 \frac{W}{m \cdot K}$$

for example

$$2 \frac{W}{m \cdot K} and 40 \frac{W}{m \cdot K} ,$$

for mullite (about 70% aluminum oxide) or

$$6 \frac{W}{m \cdot K}$$

for aluminum oxide of >99.7% purity. The thermal conductivity of the layer of fiber composite ceramic is between

$$0.5 \frac{W}{m \cdot K} and 10 \frac{W}{m \cdot K} ,$$

preferably between

$$1 \frac{W}{m \cdot K} and 5 \frac{W}{m \cdot K} .$$

These values are at 25° C. The ratio of the thermal conductivity of the monolithic ceramic and the fiber composite ceramic in the composite structure is advantageously between 1:1 and 10:1, in particular between 1:1 and 5:1.

The pressure reactor is designed for the following pressure ranges; advantageously 0.1 bar_{abs}-100 bar_{abs}, preferably 1 bar_{abs}-10 bar_{abs}, more preferably 1.5 bar_{abs}-5 bar_{abs}.

The pressure difference between the reaction chamber and the heating chamber is advantageously from 0 bar to 100 bar, preferably from 0 bar to 10 bar, more preferably from 0 bar to 5 bar.

The heat transfer coefficient of the multilayered composite tube according to the invention is advantageously

$$> 500 \frac{W}{{m}^{2} K} ,$$

preferably

$$> 1000 \frac{W}{{m}^{2} K} ,$$

more preferably

$$> 2000 \frac{W}{{m}^{2} K} ,$$

in particular

$$> 3000 \frac{W}{{m}^{2} K} .$$

The procedure for determining the heat transfer coefficient is known to a person skilled in the art (Chapter Cb: Wärmedurchgang, VDI-Wärmeatlas, 8th Edition, 1997). According to this definition:

$${k}_{loc} = \frac{1}{{R}_{w} \cdot A} , wherein$$

$${R}_{w} = {\sum }_{j = 1}^{n} {( \frac{δ}{λ \cdot {A}_{m}} )}_{j}$$

$${A}_{m , j} = {( \frac{{A}_{1} - {A}_{2}}{ln \frac{{A}_{1}}{{A}_{2}}} )}_{j}$$

The symbols have the following meanings:

R_{w}: heat transfer resistance of a multilayered cylindrical wall in

$$\frac{K}{W} ,$$

k_{loc}: heat transfer coefficient of a multilayered cylindrical wall in

$$\frac{W}{{m}^{2} K} ,$$

A: cylindrical wall area in m²,

λ: thermal conductivity in a homogenous layer in

$$\frac{W}{m K} ,$$

δ: thickness of a homogenous layer in m,

n: number of layers in a multilayered cylindrical wall,

the indices:

1: inside of a cylindrical layer,

2: outside of a cylindrical layer,

m: average area

The multilayered composite tube according to the invention may have a variable cross section and a variable wall thickness over its length. For example, the multilayered composite tube may widen or narrow in a funnel-like manner in the flow direction of the gas.

At the two ends of the multilayered composite tube the boundary region of the outer layer may advantageously be sealed. The sealed ends serve as transitions to the gastight connection of the composite tube to metallic gas-conducting conduits, distributors, collectors or passages through the shell of the surrounding heating chamber.

Employable nonporous monolithic oxide ceramics include all oxidic ceramics known to a person skilled in the art, in particular oxide ceramics analogous to those described in Informationszentrum Technische Keramik (IZTK): Brevier technische Keramik. Fahner Verlag, Lauf (2003). Preference is given to nonporous monolithic oxide ceramics comprising at least 99% by weight of Al₂O₃ and/or mullite. Employable nonporous ceramics include in particular Haldenwanger Pythagoras 1800Z™ (mullite), Alsint 99.7™ or Friatec Degussit® AL23 (aluminum oxide).

The fiber composite materials are characterized by a matrix of ceramic particles between which ceramic fibers, especially long fibers, are embedded as a winding body or as a textile.

They are called fiber-reinforced ceramic, composite ceramic or else fiber ceramic. Matrix and fiber may in principle consist of any known ceramic materials, and carbon is also treated as a ceramic material in this connection.

“Oxidic fiber composite ceramic” is to be understood as meaning a matrix of oxidic ceramic particles comprising ceramic, oxidic and/or nonoxidic fibers.

Preferred oxides of the fibers and/or the matrix are oxides of an element from the group of: Be, Mg, Ca, Sr, Ba, rare earths, Th, U, Ti, Zr, Hf, V, Nb, Ta, Cr, Mo, W, Mn, Fe, Co, Ni, Zn, B, Al, Ga, Si, Ge, Sn, Li, Na, K, Rb, Cs, Re, Ru, Os, Ir, Pt, Rh, Pd, Cu, Ag, Au, Cd, In, TI, Pb, P, As, Sb, Bi, S, Se, Te, and mixtures of these oxides.

The mixtures are advantageously suitable both as material for the fiber and for the matrix. Fiber and matrix need generally not be made of the same material.

In principle, not just binary mixtures but also tertiary and higher mixtures are suitable and of significance. In a mixture, the individual constituents may occur in an equimolar amount, but advantageous mixtures are those that have a significantly different concentration of the individual constituents of the mixture, up to and including dopings in which one component occurs in concentrations of <1%.

Particularly advantageous mixtures are as follows: binary and ternary mixtures of aluminum oxide, zirconium oxide and yttrium oxide (e.g. zirconium oxide-reinforced aluminum oxide); mixtures of silicon carbide and aluminum oxide; mixtures of aluminum oxide and magnesium oxide (MgO spinel); mixtures of aluminum oxide and silicon oxide (mullite); mixture of aluminum silicates and magnesium silicates, ternary mixture of aluminum oxide, silicon oxide and magnesium oxide (cordierite); steatite (magnesium silicate); zirconium oxide-reinforced aluminum oxide; stabilized zirconium oxide (ZrO₂): stabilizers in the form of magnesium oxide (MgO), calcium oxide (CaO) or yttrium oxide (Y₂O₃), other stabilizers used also optionally include cerium oxide (CeO₂), scandium oxide (ScO₃) or ytterbium oxide (YbO₃); and also aluminum titanate (stoichiometric mixture of aluminum oxide and titanium oxide); silicon nitride and aluminum oxide (silicon aluminum oxynitride SIALON).

Zirconium oxide-reinforced aluminum oxide used is advantageously Al₂O₃ with 10 to 20 mol % of ZrO₂. ZrO₂ can advantageously be stabilized using 10 to 20 mol % of CaO, preferably 16 mol %, 10 to 20 mol % of MgO, preferably 16, or 5 to 10 mol % of Y₂O₃, preferably 8 mol % (“fully stabilized zirconium oxide”), or 1 to 5 mol % of Y₂O₃, preferably 4 mol % (“partly stabilized zirconium oxide”). An advantageous ternary mixture is, for example, 80% Al₂O₃, 18.4% ZrO₂ and 1.6% Y₂O₃.

As well as the materials mentioned (mixtures and individual constituents), fibers of basalt, boron nitride, tungsten carbide, aluminum nitride, titanium dioxide, barium titanate, lead zirconate titanate and/or boron carbide in an oxidic ceramic matrix are also conceivable.

To obtain a desired reinforcement by the at least two layers the fibers of the fiber-reinforced ceramic may be arranged radially circumferentially and/or crossing one another on the first layer of the nonporous ceramic.

Useful fibers include reinforcing fibers that are covered by the classes of oxidic, carbidic, nitridic fibers or C fibers and SiBCN fibers. More particularly, the fibers of the ceramic composite material are aluminum oxide, mullite, silicon carbide, zirconium oxide and/or carbon fibers. Mullite consists of solid solutions of aluminum oxide and silicon oxide. Preference is given to the use of fibers of oxide ceramic (Al₂O₃, SiO₂, mullite) or of nonoxide ceramic (C, SiC).

It is advantageously possible to use creep-resistant fibers, i.e. fibers that, within the creep range within the temperature range up to 1400° C. have a minimal increase, if any, in lasting deformation over time, i.e. tendency to creep. The 3M company indicates the following threshold temperatures for a permanent elongation of 1% after 1000 hours under a tensile stress of 70 MPa for NEXTEL fibers: NEXTEL 440: 875° C., NEXTEL 550 and NEXTEL 610: 1010° C., NEXTEL 720: 1120° C. (Reference: Nextel™ Ceramic Textiles Technical Notebook, 3M, 2004).

The fibers advantageously have a diameter between 10 and 12 μm. They are advantageously interwoven typically in plain weave or satin weave to give textile sheets, knitted to form hoses or wound around a form as fiber bundles. For production of the ceramic composite system, the fiber bundles or weaves are infiltrated, for example, with a slip comprising the components of the later ceramic matrix, advantageously Al₂O₃ or mullite (Schmücker, M. (2007), Faserverstärkte oxidkeramische Werkstoffe, Materialwissenschaft and Werkstofftechnik, 38(9), 698-704). Heat treatment at >700° C. ultimately gives rise to a high-strength composite structure composed of the ceramic fibers and the ceramic matrix with a tensile strength of advantageously >50 MPa, preferably >70 MPa, further preferably >100 MPa, especially >120 MPa.

The employed ceramic fiber composite material is preferably SiC/Al₂O₃, SiC/mullite, C/Al₂O₃, C/mullite, Al₂O₃/Al₂O₃, Al₂O₃/mullite, mullite/Al₂O₃ and/or mullite/mullite. The material before the slash here denotes the fiber type and the material after the slash the matrix type. Matrix systems used for the ceramic fiber composite structure may also be siloxanes, Si precursors and a wide variety of different oxides, for example including zirconium oxide. Preferably, the ceramic fiber composite material comprises at least 99% by weight of Al₂O₃ and/or mullite.

In the present invention it is preferable to employ fiber composite materials based on oxide ceramic fibers, for example 3M™ NEXTEL™ 312, NEXTEL™ 440, NEXTEL™ 550, NEXTEL™ 610 or NEXTEL™ 720. Particular preference is given to using NEXTEL™ 610 and/or NEXTEL™ 720.

The matrix has a fill level of fibers (proportion by volume of the fibers in the composite structure) of 20% to 40%; the total solids content of the composite structure is between 50% and 80%. Fiber composite ceramics based on oxidic ceramic fibers are chemically stable in an oxidizing and in a reducing gas atmosphere (i.e. no change in weight after storage in air at 1200° C. over 15 h (reference: Nextel™ Ceramic Textiles Technical Notebook, 3M, 2004)) and are thermally stable to above 1300° C. Fiber composite ceramics have a pseudo-ductile deformation behavior. They are thus resistant to thermal shock and have quasi-tough fracture characteristics. Thus, there are signs of the failure of a component before it fractures.

The fiber composite material advantageously has an open porosity c of more than 5% to less than 50%, preferably of more than 10% to less than 30%; it is accordingly not gastight according to the definition in DIN 623-2.

The fiber composite material advantageously has a long-term use temperature of up to 1500° C., preferably up to 1400° C., more preferably up to 1300° C.

The fiber composite material advantageously has a strength >50 MPa, preferably >70 MPa, more preferably >100 MPa, especially >120 MPa.

The fiber composite material advantageously has a yield point of elastic deformation of 0.2% to 1%.

The fiber composite material advantageously has a thermal shock resistance according to DIN EN 993-11. The thermal shock resistance of the composite tube according to the invention is generally more than 50 K/h, preferably more than 300 K/h, particularly preferably more than 500 K/h.

The depressions according to the invention preferably have a depth of 0.5 to 2 mm.

The inner surface of the composite tube according to the invention is preferably provided with depressions preferably to an extent of 10% to 95%, particularly preferably 50% to 90%, based on the total inner surface area of the composite tube.

In a preferred embodiment the depressions in the composite tube according to the invention have a construction that is circular in cross section and have a (maximum) diameter of 2 mm to 30 mm.

The inner layer of the composite tube according to the invention preferably has a minimum layer thickness of 0.5 mm to 45 mm, preferably of 1 mm to 25 mm, particularly preferably of 2 mm to 15 mm.

The fiber composite material advantageously has a coefficient of thermal expansion [ppm/K] of 4 to 8.5.

The fiber composite material advantageously has a thermal conductivity of 0.5 to

$$5 \frac{W}{m \cdot K} .$$

The ceramic fiber composite material may be produced by CVI (chemical vapor infiltration) methods, pyrolysis, especially LPI (liquid polymer infiltration) methods, or by chemical reaction such as LSI (liquid silicon infiltration) methods.

The sealing of the two ends or one end of the multilayered composite tube may be performed in numerous ways:

for example, a seal can be achieved by infiltration or coating of the outer layer or of the inner layer of fiber composite ceramic or nonporous monolithic ceramic with a polymer, a nonporous ceramic, pyrolytic carbon and/or a metal. The sealed regions serve as sealing surfaces. This variant may be employed up to a temperature range of <400° C. The composite tube is advantageously coated only in the boundary region with the metallic connecting piece. “Boundary region” means the last section before the transition to another material, preferably to a metallic material, having a length corresponding to 0.05 to 10 times the internal diameter of the composite tube, preferably corresponding to 0.1-5 times the internal diameter, in particular corresponding to 0.2-2 times the internal diameter. The thickness of the impregnation advantageously corresponds to the total layer thickness of the fiber composite ceramic in the boundary region. Processes for impregnation are known to a person skilled in the art.

The present invention accordingly comprises a multilayered composite tube comprising at least two layers, namely a layer of nonporous monolithic ceramic, preferably oxide ceramic, and a layer of fiber composite ceramic, preferably oxidic fiber composite ceramic, wherein the outer layer of the composite tube is impregnated or coated with polymer, nonporous ceramic, (pyrolytic) carbon and/or metallic material in the boundary region before the transition to another material, preferably metallic material.

Another possible way of effecting sealing advantageously comprises attaching to the boundary region of the multilayered composite tube a sleeve of metal which is arranged between the inner and the outer layer in regions using an overlap joint (5). The sleeve of metal advantageously comprises one or more of the following materials: chromium, titanium, molybdenum, nickel steel 47Ni, alloy 80Pt20Ir, alloy 1.3981, alloy 1.3917 or a trimetal copper/Invar/copper. The ratio of the length of the overlap joint (5) to the internal diameter of the composite tube is advantageously in the range from 0.05 to 10, preferably from 0.1 to 5, in particular from 0.2 to 2. In this range the sleeve of metal is gastightly joined to the outside of the inner layer by means of joining techniques known to a person skilled in the art (Informationszentrum Technische Keramik (IZTK): Brevier technische Keramik, Fahner Verlag, Lauf (2003)). The outer layer is joined to the sleeve of metal by an atomic-level join. The length of the ceramic overlap, i.e. the region comprising outer layer and metallic sleeve without inner layer, is advantageously from 0.05 times to 10 times, preferably from 0.1 times to 5 times, in particular from 0.2 times to 2 times, the internal diameter of the composite tube.

The present invention accordingly comprises a multilayered composite tube comprising at least two layers, namely an inner layer of nonporous monolithic ceramic, preferably oxide ceramic, and an outer layer of fiber composite ceramic, preferably oxidic fiber composite ceramic, wherein the inner surface of the composite tube comprises a plurality of depressions oriented towards the outer wall of the composite tube and wherein a sleeve of metal disposed in regions between the inner and the outer layer is arranged at the end of the composite tube.

The present invention consequently comprises a connecting piece comprising at least one metallic gas-conducting conduit which in the longitudinal direction of the multilayered composite tube, i.e. in the flow direction of the reactants, in regions overlaps with at least two ceramic layers, wherein at least one ceramic layer comprises a nonporous monolithic ceramic, preferably oxide ceramic, and at least one other ceramic layer comprises a fiber composite ceramic, preferably oxidic fiber composite ceramic.

The present invention consequently comprises a sandwich structure in the transition region between metallic material and ceramic material comprising a metallic layer, a nonporous monolithic ceramic layer, preferably oxide ceramic, and a fiber composite ceramic layer, preferably oxide fiber composite ceramic. The metallic layer is preferably between the inner and the outer ceramic layer.

The present invention advantageously comprises a connecting piece comprising a first tube region comprising a metallic tube, for example at least one metallic gas-conducting conduit, comprising a second tube region connected to the first tube region which comprises an outer layer of fiber composite ceramic and an inner metallic layer and a third tube region connected to the second tube region which comprises a sandwich structure comprising a metallic layer, a nonporous monolithic ceramic layer and a fiber composite ceramic layer and a fourth tube region connected to the third tube region which comprises a multilayered composite tube comprising at least two layers, namely a layer of nonporous monolithic ceramic and a layer of fiber composite ceramic.

The sandwich structure of the connecting piece advantageously comprises an inner ceramic layer, an intermediate metallic layer and an outer ceramic layer. The fiber composite ceramic is advantageously the outer ceramic layer. The nonporous monolithic ceramic layer is advantageously the inner layer. Alternatively, the fiber composite ceramic is the inner ceramic layer. Alternatively, the nonporous monolithic ceramic layer is the outer layer. The fiber composite ceramic is preferably oxidic. The nonporous monolithic ceramic is preferably an oxide ceramic.

The length of the first tube region is more than 0.05 times, preferably more than 0.1 times, in particular more than 0.2 times, the internal diameter of the multilayered composite tube; the length of the first tube region is advantageously less than 50% of the total length of the composite tube.

The length of the second tube region is from 0.05 times to 10 times, preferably from 0.1 times to 5 times, in particular from 0.2 times to 2 times, the internal diameter of the multilayered composite tube.

The length of the third tube region is from 0.05 times to 10 times, preferably from 0.1 times to 5 times, in particular from 0.2 times to 2 times, the internal diameter of the composite tube.

In the third tube region the wall thickness of the metallic tube, i.e. the metallic overlap, is advantageously 0.01 times to 0.5 times the total wall thickness, preferably 0.03 times to 0.3 times the total wall thickness, in particular 0.05 times to 0.1 times the total wall thickness. In the second tube region the wall thickness of the ceramic overlap is advantageously 0.05 times to 0.9 times the total wall thickness, preferably 0.05 times to 0.5 times the total wall thickness, in particular 0.05 times to 0.25 times the total wall thickness. In the second tube region the wall thickness of the sleeve is advantageously 0.05 times to 0.9 times the total wall thickness, preferably 0.05 times to 0.5 times the total wall thickness, in particular 0.05 times to 0.025 times the total wall thickness.

The thickness of the layer of monolithic ceramic is advantageously from 0.5 mm to 45 mm, preferably from 1 mm to 25 mm, particularly preferably from 3 mm to 15 mm. The thickness of the layer of oxidic fiber composite ceramic is advantageously from 0.5 mm to 5 mm, preferably from 0.5 mm to 3 mm.

Another possible way of effecting sealing advantageously comprises attaching to the end of the multilayered composite tube a sleeve of metal whose inner surface and outer surface are in regions joined to the inner layer and to the outer layer. The joining to the inner layer is effected gastightly with joining techniques known to a person skilled in the art (Informationszentrum Technische Keramik (IZTK): Brevier technische Keramik, Fahner Verlag, Lauf (2003)). The join to the outer layer is an atomic-level join.

The present invention advantageously comprises a connecting piece comprising a first tube region comprising a metallic tube, for example at least one metallic gas-conducting conduit, comprising a second tube region connected to the first tube region which comprises an outer ceramic layer and an inner metallic layer and a third tube region connected to the second tube region which comprises a sandwich structure comprising an inner metallic layer, an intermediate ceramic layer and an outer ceramic layer, wherein one of the ceramic layers comprises a nonporous monolithic ceramic layer and the other ceramic layer comprises a fiber composite ceramic layer, and a fourth tube region connected to the third tube region which comprises a multilayered composite tube comprising at least two layers, namely a layer of nonporous monolithic ceramic and a layer of fiber composite ceramic.

The fiber composite ceramic is advantageously the outer ceramic layer. The nonporous monolithic ceramic layer is advantageously the inner layer. Alternatively, the fiber composite ceramic is the inner ceramic layer. Alternatively, the nonporous monolithic ceramic layer is the outer layer. The fiber composite ceramic is preferably oxidic. The nonporous monolithic ceramic is preferably an oxide ceramic.

The length of the first tube region is more than 0.05 times, preferably more than 0.1 times, in particular more than 0.2 times, the internal diameter of the multilayered composite tube; the length of the first tube region is advantageously less than 50% of the total length of the composite tube.

The length of the second tube region is from 0.05 times to 10 times, preferably from 0.1 times to 5 times, in particular from 0.2 times to 2 times, the internal diameter of the multilayered composite tube.

The length of the third tube region is from 0.05 times to 10 times, preferably from 0.1 times to 5 times, in particular from 0.2 times to 2 times the internal diameter of the composite tube.

In the third tube region the wall thickness of the metallic tube, i.e. the metallic overlap, is advantageously 0.01 times to 0.5 times the total wall thickness, preferably 0.03 times to 0.3 times the total wall thickness, in particular 0.05 times to 0.1 times the total wall thickness.

In the second tube region the wall thickness of the ceramic overlap is advantageously 0.1 times to 0.95 times the total wall thickness, preferably 0.5 times to 0.95 times the total wall thickness, in particular 0.8 times to 0.95 times the total wall thickness. In the second tube region the wall thickness of the sleeve is advantageously 0.05 times to 0.9 times the total wall thickness, preferably 0.05 times to 0.5 times the total wall thickness, in particular 0.05 times to 0.2 times the total wall thickness.

The thickness of the layer of monolithic ceramic is advantageously from 0.5 mm to 45 mm, preferably from 1 mm to 25 mm, particularly preferably from 3 mm to 15 mm. The thickness of the layer of oxidic fiber composite ceramic is advantageously from 0.5 mm to 5 mm, preferably from 0.5 mm to 3 mm.

The present invention advantageously comprises a connecting piece comprising a first tube region comprising a metallic tube, for example at least one metallic gas-conducting conduit, comprising a second tube region connected to the first tube region which comprises a sandwich structure comprising an inner ceramic layer, an intermediate metallic layer and an outer ceramic layer, wherein one of the ceramic layers comprises a nonporous monolithic ceramic layer and the other ceramic layer comprises a fiber composite ceramic layer, and a third tube region connected to the second tube region which comprises a multilayered composite tube comprising at least two layers, namely a layer of nonporous monolithic ceramic and a layer of fiber composite ceramic.

The fiber composite ceramic is advantageously the inner ceramic layer. The nonporous monolithic ceramic layer is advantageously the outer layer. Alternatively, the fiber composite ceramic is the outer ceramic layer. Alternatively, the nonporous monolithic ceramic layer is the inner layer. The fiber composite ceramic is preferably oxidic. The nonporous monolithic ceramic is preferably an oxide ceramic.

The length of the second tube region is from 0.05 times to 10 times, preferably from 0.1 times to 5 times, in particular from 0.2 times to 2 times, the internal diameter of the multilayered composite tube.

In the second tube region the wall thickness of the metallic tube, i.e. the metallic overlap, is advantageously 0.01 times to 0.5 times the total wall thickness, preferably 0.03 times to 0.3 times the total wall thickness, in particular 0.05 times to 0.1 times the total wall thickness.

In the second tube region the wall thickness of the ceramic overlap is advantageously 0.1 times to 0.95 times the total wall thickness, preferably 0.5 times to 0.95 times the total wall thickness, in particular 0.8 times to 0.95 times the total wall thickness. In the second tube region the wall thickness of the sleeve is advantageously 0.05 times to 0.9 times the total wall thickness, preferably 0.05 times to 0.5 times the total wall thickness, in particular 0.05 times to 0.2 times the total wall thickness.

The thickness of the layer of monolithic ceramic is advantageously from 0.5 mm to 45 mm, preferably from 1 mm to 25 mm, particularly preferably from 3 mm to 15 mm. The thickness of the layer of oxidic fiber composite ceramic is advantageously from 0.5 mm to 5 mm, preferably from 0.5 mm to 3 mm.

The present invention advantageously comprises a connecting piece comprising a first tube region comprising a metallic tube, for example at least one metallic gas-conducting conduit, comprising a second tube region connected to the first tube region which comprises a sandwich structure comprising an inner ceramic layer and an intermediate ceramic layer and an outer metallic layer, wherein one of the ceramic layers comprises a nonporous monolithic ceramic layer and the other ceramic layer comprises a fiber composite ceramic layer, and a third tube region connected to the second tube region which comprises a multilayered composite tube comprising at least two layers, namely a layer of nonporous monolithic ceramic and a layer of fiber composite ceramic.

The fiber composite ceramic is advantageously the inner ceramic layer. The nonporous monolithic ceramic layer is advantageously the outer layer. Alternatively, the fiber composite ceramic is the outer ceramic layer. Alternatively, the nonporous monolithic ceramic layer is the inner layer. The fiber composite ceramic is preferably oxidic. The nonporous monolithic ceramic is preferably an oxide ceramic.

The length of the second tube region is from 0.05 times to 10 times, preferably from 0.1 times to 5 times, in particular from 0.2 times to 2 times, the internal diameter of the multilayered composite tube.

In the second tube region the wall thickness of the metallic tube, i.e. the metallic overlap, is advantageously 0.01 times to 0.5 times the total wall thickness, preferably 0.03 times to 0.3 times the total wall thickness, in particular 0.05 times to 0.1 times the total wall thickness.

In the second tube region the wall thickness of the ceramic overlap is advantageously 0.1 times to 0.95 times the total wall thickness, preferably 0.5 times to 0.95 times the total wall thickness, in particular 0.8 times to 0.95 times the total wall thickness. In the second tube region the wall thickness of the sleeve is advantageously 0.05 times to 0.9 times the total wall thickness, preferably 0.05 times to 0.5 times the total wall thickness, in particular 0.05 times to 0.2 times the total wall thickness.

The thickness of the layer of monolithic ceramic is advantageously from 0.5 mm to 45 mm, preferably from 1 mm to 25 mm, particularly preferably from 2 mm to 15 mm. The thickness of the layer of oxidic fiber composite ceramic is advantageously from 0.5 mm to 5 mm, preferably from 0.5 mm to 3 mm.

The multilayered composite tube is typically arranged vertically, mounted in a fixed manner at one end and mounted loosely at the other end. Preference is given to it being clamped in a fixed manner at the lower end and being arranged movably in an axial direction at the upper end. In this arrangement, the tube can undergo thermal expansion without stresses.

One variant of the solution consists of two concentric tubes. The inner tube advantageously has a tube internal diameter of 10 mm to 100 mm, preferably 15 mm to 50 mm, in particular 20 mm to 30 mm. The inner tube is advantageously open at both ends and the outer tube is advantageously closed at one end. The outer tube advantageously has a tube internal diameter of 20 mm to 1000 mm, preferably 50 mm to 800 mm, in particular 100 mm to 500 mm.

At the open boundary region the walls of the inner and outer tubes are advantageously sealed. The main reaction section is advantageously disposed in the annular space between the inner tube and the outer tube. The reactants may either be introduced into the annular space and the product stream withdrawn from the inner tube or vice versa. The feeder and discharge connections are disposed at the open tube end. The closed tube end may project loosely (without any guide) into the heating space and therein expand unhindered. This ensures that no temperature-induced stresses can arise in the axial direction. This configuration ensures that the multilayered composite tubes need only be clamped and sealed at one end in the cold state and can undergo thermal expansion unhindered at the closed end.

The present invention thus comprises a double-tube reactor for endothermic reactions, wherein the reactor comprises two multilayered composite tubes having a heat transfer coefficient of >500 W/m²/K and comprising in each case at least two layers, namely a layer of nonporous monolithic ceramic and a layer of fiber composite ceramic, wherein the one composite tube surrounds the other composite tube and the inner composite tube is open at both ends and the outer tube is closed at one end.

The fiber composite ceramic is advantageously the outer ceramic layer of the multilayered composite tube comprising two concentric tubes. The nonporous monolithic ceramic layer is advantageously the inner layer. Alternatively, the fiber composite ceramic is the inner ceramic layer. Alternatively, the nonporous monolithic ceramic layer is the outer layer. The fiber composite ceramic is preferably oxidic. The nonporous monolithic ceramic is preferably an oxide ceramic.

The double-layered structure makes it possible to combine the gastightness and heat resistance of a tube made of monolithic nonporous ceramic with the favorable failure behavior of the fiber composite ceramic (“crack before fracture”).

The apparatus according to the invention having sealed boundary regions makes it possible to achieve gastight connection of the multilayered composite tubes to the conventionally configured periphery.

It is advantageous to employ the ceramic multilayered composite tubes according to the invention for the following processes:
Production of synthesis gas by reforming of hydrocarbons with steam and/or CO₂.
Coproduction of hydrogen and pyrolysis carbon through pyrolysis of hydrocarbons.
Production of hydrocyanic acid from methane and ammonia (Degussa) or from propane and ammonia.
Production of olefins by steamcracking of hydrocarbons (naphtha, ethane, propane).
Coupling of methane to give ethylene, acetylene and benzene.

It is advantageous to employ the ceramic composite tubes according to the invention as reaction tubes in the following applications:
Reactors with axial temperature control, such as
   fluidized bed reactors,
   shell and tube reactors,
   reformers and cracking furnaces.
Jet tubes, flame tubes.
Countercurrent reactors.
Membrane reactors.
Rotary tubes for rotary tube furnaces.

The advantages of the multilayered composite tube according to the invention are hereinbelow demonstrated by comparative examples.

### EXAMPLE 1: COMPARISON OF TEMPERATURE DISTRIBUTION ON AN INVENTIVE MULTILAYERED COMPOSITE TUBE WITH DEPRESSIONS AND A MULTILAYERED COMPOSITE TUBE WITHOUT DEPRESSIONS

The temperature distribution in a steam-conducting tube was determined by numerical simulation (CFD=computational fluid dynamics). In this example a 1 m-long multilayered ceramic composite tube of 0.047 m internal diameter and tube wall thicknesses of 4 mm for the monolithic ceramic and 1.5 mm for the fiber ceramic were simulated.

The following table 3 shows the properties of the tube materials employed here.

| | | Fiber | Metal |
|---|---|---|---|
| Material data at 900° C. | Al₂O₃ | ceramic | tube |
| ρ (density, kg/m3) | 2800 | 2900 | 7600 |
| cₚ (specific heat capacity, J/kgK) | 900 | 900 | 663 |
| λ (thermal conductivity, W/mK) | 706.1*T’^{(-0.672)} | 58.9*T’^{(-0.479)} | 24 |

T’ = local temperature in ° C.

In addition to a tube with inventive depressions a tube of identical structure without depressions was simulated. In the tube with depressions 8 depressions per circumferential segment with a radius of in each case 13.8 mm and a displaced arrangement in the axial direction with a distance of 12.5 mm between the centers of the depressions were modelled.

An entry temperature of the fluid of 750° C., a mass flow of 8 kg/s and a constant outer tube wall temperature of 950° C. were specified in the simulation.

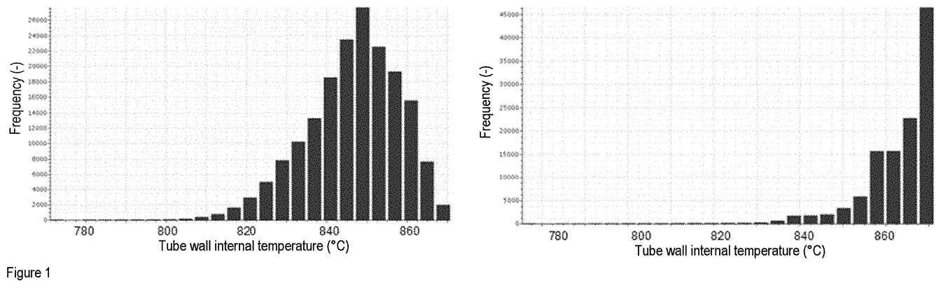

The results of the simulation are shown in FIG. 1. The frequency distribution (number of surface elements discretized in the simulation) versus the tube wall internal temperature is plotted in the left-hand panel for the inventive tube with depressions and in the right hand panel for a tube of identical construction without depressions. It is apparent that the depressions altogether reduce the average tube wall temperature and thus coking compared to the tube without depressions while simultaneously the heat flow transferred to the fluid stream increases by 14% on account of the improved heat transfer. The example also shows that the distribution of the tube wall temperature becomes broader due to the locally improved heat transfer at the depressions. This is especially advantageous because this effect reduces coking in the interior of the depressions and the structure of the depressions and the effect of the improved heat transfer is thus retained even during the process of coking.

### EXAMPLE 2: COMPARISON OF THE TEMPERATURE DISTRIBUTION ON AN INVENTIVE MULTILAYERED COMPOSITE TUBE WITH DEPRESSIONS AND A METALLIC TUBE (MATERIAL S+C CENTRALLOY® HT-E) WITH DEPRESSIONS

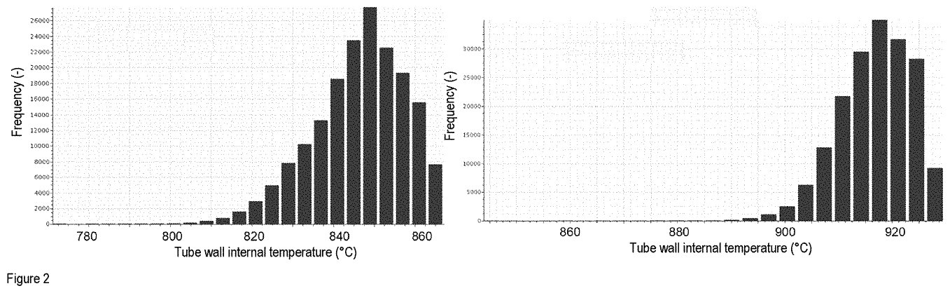

In a second example the above inventive multilayered composite tube with depressions was compared to a geometrically identical metallic tube with depressions. The results of the simulation are shown in FIG. 2. The frequency distribution (number of surface elements discretized in the simulation) versus the tube wall internal temperature is plotted in the left-hand panel for the tube according to the invention with depressions and in the right hand panel for a metallic tube of identical structure likewise with depressions of identical structure. As is shown in FIG. 2 the temperature distribution for the ceramic tube is broader. This mirrors a larger temperature difference between the depressions (low-temperature) and the remaining tube wall surface area (high-temperature) for the ceramic tube. It is thought that the poorer thermal conductivity in the ceramic tube results in this more marked temperature scattering. This result is surprising and shows that the depressions are more advantageous for a ceramic tube than for metallic tubes since for a ceramic tube coke formation is especially reduced at the depressions and the positive effect of the depressions is thus retained for longer. In the case of metallic tubes the depressions are rapidly filled by coke formation.

1. A multilayered composite tube having a heat transfer coefficient of >500 W/m²/K comprising at least two layers which in its construction over the cross section of the wall of the composite tube comprises as an inner layer a zero-open-porosity monolithic oxide ceramic surrounded by an outer layer of oxidic fiber composite ceramic, wherein this outer layer has an open porosity ε of 5%<ε<50%, and which on the inner surface of the composite tube comprises a plurality of depressions oriented towards the outer wall of the composite tube.

2. The composite tube according to claim 1, wherein the thermal shock resistance according to DIN EN 993-11 of the composite tube is greater than 50 K/h.

3. The composite tube according to claim 1, wherein the depressions have a depth of 0.5 mm to 2 mm.

4. The composite tube according to claim 1, wherein the depressions are uniformly distributed over the inner surface of the composite tube.

5. The composite tube according to claim 1, wherein the depressions are nonuniformly distributed over the inner surface of the composite tube.

6. The composite tube according to claim 1, wherein the inner surface of the composite tube is provided with depressions to an extent of 10% to 95% based on the total inner surface of the composite tube.

7. The composite tube according to claim 1, wherein the depressions are concave.

8. The composite tube according to claim 1, wherein the depressions have a construction that is circular in cross section and have a diameter of 2 mm to 30 mm.

9. The composite tube according to claim 1, wherein the total wall thickness of the composite tube is 0.5 mm to 50 mm.

10. The composite tube according to claim 1, wherein the tube internal diameter of the composite tube is 10 mm to 1000 mm.

11. The composite tube according to claim 1, wherein the employed oxidic fiber composite ceramic is SiC/Al₂O₃, SiC/mullite, C/Al₂O₃, C/mullite, Al₂O₃/Al₂O₃, Al₂O₃/mullite, mullite/Al₂O₃ and/or mullite/mullite.

12. The composite tube according to claim 1, wherein the composite tube contains two layers, including an inner layer and an outer layer, wherein the inner layer is constructed from nonporous monolithic oxide ceramic and the outer layer is constructed from oxidic fiber composite ceramic.

13. The composite tube according to claim 1, wherein the composite tube has a structure in which the nonporous monolithic oxide ceramic is covered by oxidic fiber composite ceramic.

14. The composite tube according to claim 1, wherein the inner layer has a minimum layer thickness of 0.5 mm to 45 mm.

15. A method of using the composite tube according to claim 1, the method comprising using the composite tube in the production of synthesis gas by reforming of hydrocarbons with steam and/or carbon dioxide, coproduction of hydrogen and pyrolysis carbon by pyrolysis of hydrocarbons, production of hydrocyanic acid from methane and ammonia or from propane and ammonia, production of olefins by steamcracking of hydrocarbons and/or coupling of methane to ethylene, acetylene and to benzene.

16. A method of using the composite tube according to claim 1, the method comprising using the composite tube as a reaction tube in reactors with axial temperature control, countercurrent reactors, membrane reactors, jet tubes, flame tubes and/or rotary tubes for rotary tube furnaces.

17. A process for producing the multilayered composite tube according to claim 1, the process comprising impressing the depressions by pressing processes.

18. The composite tube according to claim 1, wherein the outer layer has an open porosity ε of 10%<ε<30%.
